(19) United States
(12) Patent Application Publication
(10) Pub. No.: US 2007/0224591 A1
(43) Pub. Date: Sep. 27, 2007

(54) Rotation-based microsampler, system and method of using the same

(75) Inventors: John Yupeng Gui, Niskayuna, NY (US); Wei-Cheng Tian, Clifton Park, NY (US); Atanu Phukan, Bangalore (IN); Shashi Thutupalli, Bangalore (IN); Victor Samper, Bavaria (DE)

Correspondence Address: GENERAL ELECTRIC COMPANY;GLOBAL RESEARCH, PATENT DOCKET RM. BLDG. K1-4A59, NISKAYUNA, NY, 12309, US

(73) Assignee: General Electric Company

(21) Appl. No.: 11/389,586

(22) Filed: Mar. 27, 2006

(51) Int. Cl. C12Q 1/00; C12M 3/00

(52) U.S. Cl. 435/4; 435/287.1

(57) ABSTRACT

A microsampler disc for use in the analysis of agents is described as including a plurality of microstructures configured and spaced to promote movement of a fluidic medium containing agents radially outwardly and promote filtering of one species of agents from other species of agents. An analysis system using the microsampler disc is also described. A method for separating one species of agent from one or more other species of agents is described as including introducing a fluidic medium containing at least one species of agents to a microsampler disc having a plurality of microstructures, rotating the microsampler disc to promote movement of the fluidic medium outwardly, collecting the at least one species of agent in a specific set of detection zones, and analyzing the at least one species of agent.

### BACKGROUND

The invention relates generally to the micro-particle sampling. More particularly, the invention relates to sampling micro-particles utilizing centrifugal forces and filtering techniques and mechanisms.

The accurate detection of biological and/or chemical species within a fluidic medium is of importance for numerous industries. Examples of such a need include, but are not limited to, (a) ascertaining a specific environmental pollutant from samples taken from an environmental site; (b) determining a type of pathogen that has been released into an ecosystem so as to determine appropriate countermeasures; and (c) separating target biological entities, such as cells, DNA, RNA, and proteins, from unwanted biological species for biological studies and clinical diagnosis.

Various techniques are currently used for cell separations, such as, for example, mechanical sieves having different sized pores, electrophoreses, di-electrophoresis, magnetic beads, and gravity-enabled separations. These techniques are typically limited to sorting two types of particles. Sorting more than two types of particles remains a challenge. Further, known separation techniques suffer from clogging issues, where one of the types of particles creates an obstruction that further clogs up the other type(s) of particles. This forces a need for higher sampling rates.

### BRIEF DESCRIPTION OF THE DRAWINGS

FIG. 1 is a top view of a microsampler disc constructed in accordance with an exemplary embodiment of the invention.

FIG. 2 is an exploded view of a portion of the microsampler disc of FIG. 1 taken within circle II.

FIG. 3 is a cross-sectional view taken along line III-III of FIG. 1.

FIG. 4 is a schematic view of a microfluidic channel and a first micropillar ring of the microsampler disc of FIG. 1.

FIG. 5 is another schematic view of a microfluidic channel and a first micropillar ring of the microsampler disc of FIG. 1.

FIG. 6 is a close-up schematic view from the top of a portion of a first micropillar ring of the microsampler disc of FIG. 1.

FIG. 7 is a side view of first and second micropillars of the microsampler disc of FIG. 1.

FIG. 8 is a schematic top view of a microsampler disc constructed in accordance with an exemplary embodiment of the invention.

FIG. 9 is a schematic view from the top of a microsampler disc constructed in accordance with an exemplary embodiment of the invention.

FIG. 10 is a schematic view from the top of a microsampler disc constructed in accordance with an exemplary embodiment of the invention.

FIG. 11 is an exploded view of a portion of the microsampler disc of FIG. 10 taken within circle XI.

FIG. 12 is an exploded view of a portion of the microsampler disc of FIG. 10 taken within circle XII.

FIG. 13 schematically illustrates motion of microparticles due to a clockwise fluid flow in accordance with an embodiment of the invention.

FIG. 14 schematically illustrates motion of microparticles due to a counter-clockwise fluid flow in accordance with an embodiment of the invention.

FIGS. 15A-B are schematic views from the top of a microsampler disc constructed in accordance with exemplary embodiments of the invention.

FIG. 15C is a view of a portion of the microsampler disc of FIGS. 15A-B.

FIG. 16 is a schematic view from the top of a microsampler disc constructed in accordance with an exemplary embodiment of the invention.

FIG. 17 schematically illustrates an analyzing system in accordance with an embodiment of the invention.

FIG. 18 illustrates process steps for separating one species of analytes from other species of analytes in accordance with an embodiment of the invention.

### SUMMARY

Embodiments of the invention are directed to a microsampler and to a system and a method for separating one species of analytes from other species of analytes.

In accordance with some embodiments, there is described a microsampler disc for use in the detection of agents. The microsampler disc includes a plurality of microstructures configured and spaced to promote movement of a fluidic medium containing agents radially outwardly and promote filtering of one species of agents from other species of agents.

In accordance with some embodiments, there is described a microsampler device that includes a microsampler disc and a second disc. The microsampler disc includes a concentric set of first micropillars and a concentric set of second micropillars. The first micropillars are at least one of a first size and a first spacing between adjacent first micropillars, and the set of second micropillars are at least one of a second size and a second spacing between adjacent second micropillars. The second disc rests upon the concentric sets of first and second micropillars.

In accordance with some embodiments, there is described a system for analyzing agents in a fluidic medium. The system includes a microsampler device comprising a microsampler disc having a plurality of microstructures for filtering a first species of agents from other species of agents. The system also includes a rotational mechanism for rotating the microsampler device to exert a centrifugal force on the fluidic medium and a detector for detecting the presence of one or more specific species of agents.

In accordance with some embodiments, there is described a method of detecting at least one species of agent in a fluidic medium. The method includes introducing a fluidic medium containing two or more species of agents to a microsampler disc. The microsampler disc includes a plurality of microstructures for filtering the at least one species of agents from the remainder of the fluidic medium. The method further includes rotating the microsampler disc to promote movement of the fluidic medium outwardly, collecting the at least one species of agents in a specific set of detection zones, and analyzing the at least one species of agents.

These and other advantages and features will be more readily understood from the following detailed description of preferred embodiments of the invention that is provided in connection with the accompanying drawings.

### DETAILED DESCRIPTION OF EXEMPLARY EMBODIMENTS

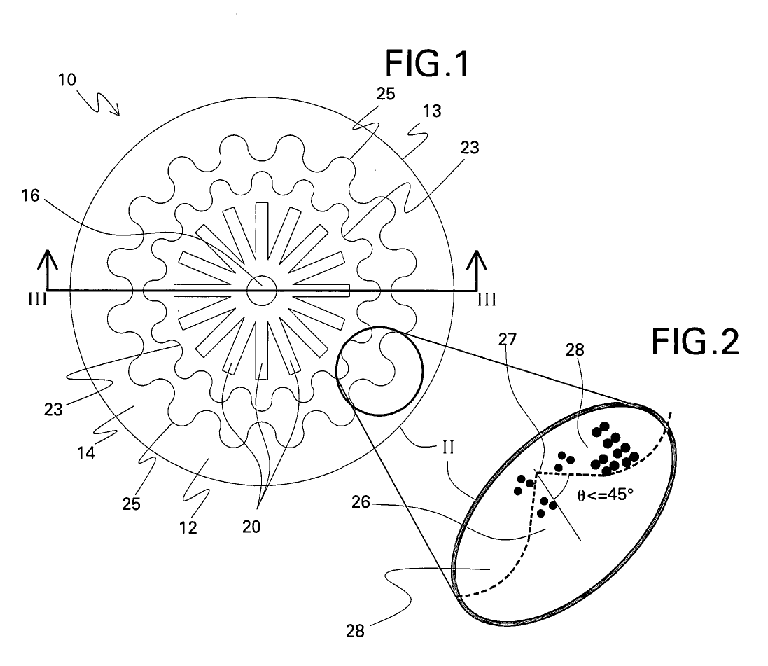

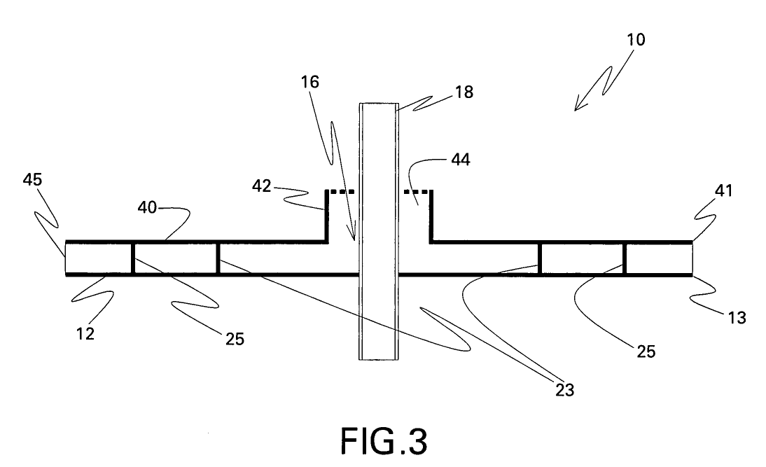

Referring to FIGS. 1-7, a microfluidic sampler 10 is shown. Specifically, FIG. 3 shows the microfluidic sampler 10 including a first plate or disc 12 and a second plate or disc 40. As shown, the discs 12, 40 extend out to coextensive edges 13, 41, respectively. The space between the edges 13, 41 may be sealed with a seal 45 or may be left unsealed. The disc 40 is positioned above the disc 12, each of the discs 12, 40 being separated from the other by a plurality of micropillars in a first set of rings 23 and by another plurality of micropillars in a second set of rings 25. It should be appreciated that the rings of micropillars 23, 25 may attach to a surface 14 of first disc 12 and extend toward second disc 40, or attach to second disc 40 and extend toward first disc 12. The second disc 40 may rest upon a plurality of microstructures situated on the first disc 12.

The first disc 12 may include an opening 16 through which a rotatable shaft 18 may extend. The rotatable shaft 18 would rotate, translating its rotation to the microfluidic sampler 10. The second disc 40 includes an annular wall 42 that defines a reservoir 44. The optional rotatable shaft 18 may extend up through the reservoir 44. A fluidic medium, which may include one or more species of pathogens or agents, may be introduced into the reservoir 44 and through the translated rotational motion move radially outwardly between the two discs 12, 40.

Each agent in the fluidic medium will become attached to a tag or marker and to a microbead or microparticle of a specific size. The attachment of the agents and tags to the microbeads may be brought about using antibody chemistries. All of the agents of a single species will include the same type of tag and the same size microbead. Agents of one species will have a microbead differently sized than agents of another species.

With specific reference to FIGS. 1-5, next will be described numerous microstructures useful in separating one species of agents from another species found within the fluidic medium. Extending radially outward from the opening 16 is a plurality of microchannels 20, which serve as conduits for the fluidic medium. Radially exterior to the microchannels 20 is the first set of rings 23 and then the second set of rings 25. Each set of rings 23, 25 is formed of a plurality of micropillars. Specifically, set 23 includes a plurality of micropillars 30. The micropillars 30 are sized and positioned so as to capture one species of agent within the detection zones 28 while allowing the other species of agents to filter through the micropillars 30 to a subsequent ring of micropillars.

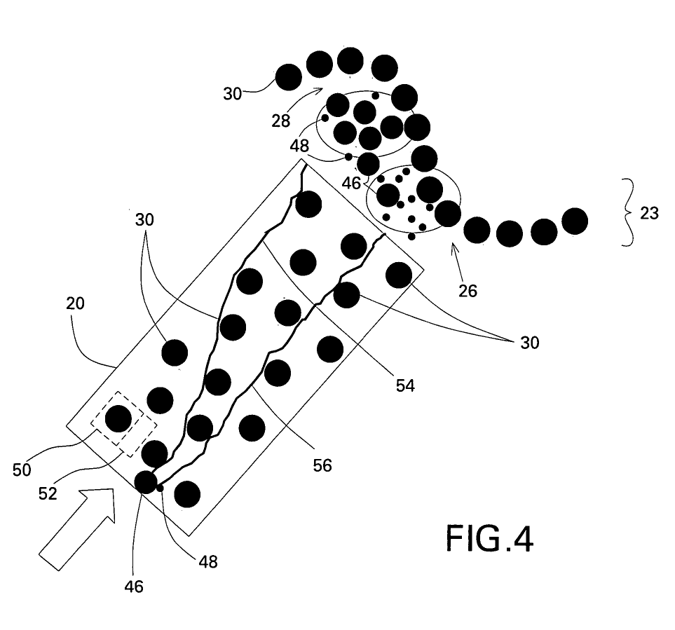

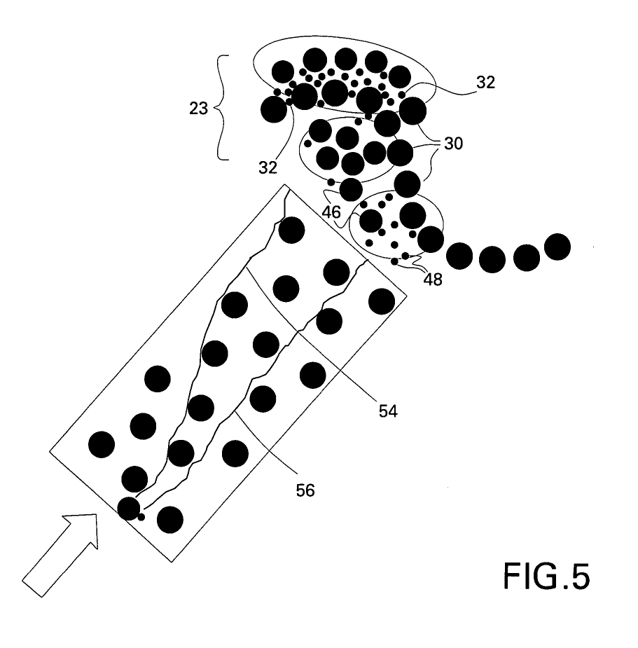

The micropillars 30 in set 23 may be positioned in one ring (FIG. 4) or more rings (FIG. 5). It should be appreciated that, while two sets 23, 25 of rings are illustrated, there may be a higher number of sets of rings, such as, for example, ten, twenty, fifty, or even one hundred sets of rings. Each of the rings within either of the sets 23, 25 are configured such that the micropillars form alternating filtering zones 26 and detection zones 28. With specific reference to FIG. 2, one embodiment includes a filtering zone 26 that has a pair of lines of micropillars that join at an abutment point 27 and that are substantially orthogonal to one another. Other embodiments include filtering zones 26 that are rounded or squared off or funnel-shaped. It should be appreciated that the filtering zones 26 may be formed in any configuration provided that configuration enhances, or at the least does not detain, the movement of one species of agents to the detection zones 28 and the movement of other species of agents to subsequent rings of micropillars.

Referring now to FIG. 4, a single microchannel 20 extending toward a first set of micropillar rings 23 is shown. A plurality of micropillars 30 is positioned within the microchannel 20. These micropillars 30 serve to pre-filter one agent species from another prior to reaching the sets of rings 23, 25. As shown, one particle 46 (including an agent from one species and a microbead) is positioned in a first region 50 immediately surrounding a micropillar 30. Further, another particle 48 (including an agent from another species and a microbead) is positioned in a second region 52 between two adjacent micropillars 30. The relative positions of particles 46 and 48, as well as the rotation of the microfluidic sampler 10 and the positioning of the micropillars 30 in the microchannel 20 all serve to enhance particle 48 to take a first path 54 and the particle 46 to take a second path 56 through the microchannel 20. Through such pre-filtering, it may be possible to obtain as much as eighty percent (80%) separation of particles 48 from particles 46.

As the particles 46, 48 are transported out of the microchannel 20 toward the set of rings 23, the particles 46 are forced into the detection zones 28. The particles 46 may go initially into the detection zones 28 or they may be forced into the filtering zones 26 and deflected into the detection zones 28. The spacing of the micropillars 30 in the set of rings 23 is such that the particles 46 become trapped but the particles 48 continue through to a subsequent set of rings 25.

The second set of rings 25 may include a plurality of micropillars 32 which are smaller in size than the micropillars 30. The micropillars 32 may be sized and positioned to trap a second agent species and allow other agent species to filter through in the same manner as described above with regard to the set of rings 23. Subsequent sets of rings may be positioned radially outward on the microfluidic sampler 10 for trapping other species of agents. It should be appreciated that each set of rings may be positioned close to adjacent sets of rings, or instead may be positioned at a distance from adjacent sets of rings.

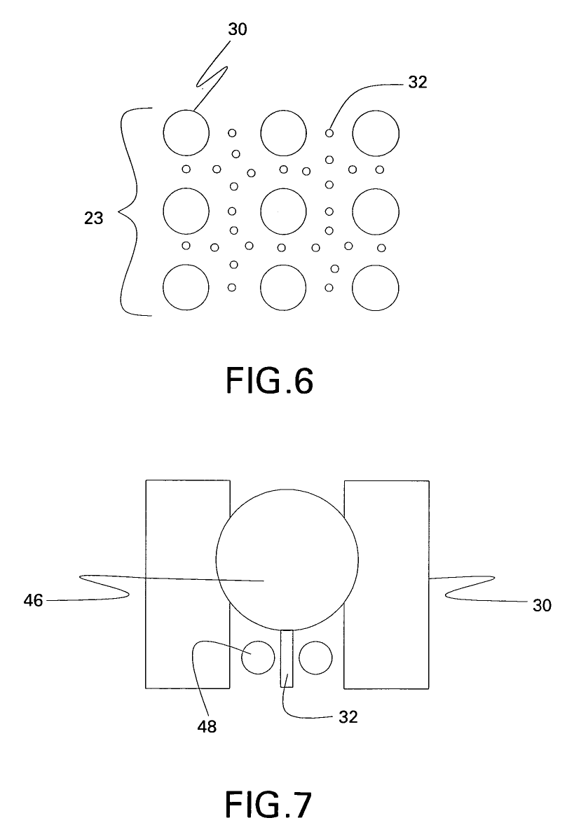

It should be appreciated that an accumulation of particles 46 may be sufficiently dense as to block particles 48 from moving into and through the filtering zones 26. In one embodiment, micropillars 32 may be positioned between micropillars 30 (FIG. 6). As shown in FIG. 7, the micropillars 32 are shorter in height than the micropillars 30, thus allowing particles 46 to rest on top of the micropillars 32 and leaving a pathway for particles 48 to move underneath the particles 46 and through the micropillars 30, 32.

It should also be appreciated that the presence of microchannels 20 is optional, and any suitable microfluidic sampler may or may not include microchannels 20. Further, it should be appreciated that second disc 40 may rest upon other supporting microstructures other than the micropillars 30, 32.

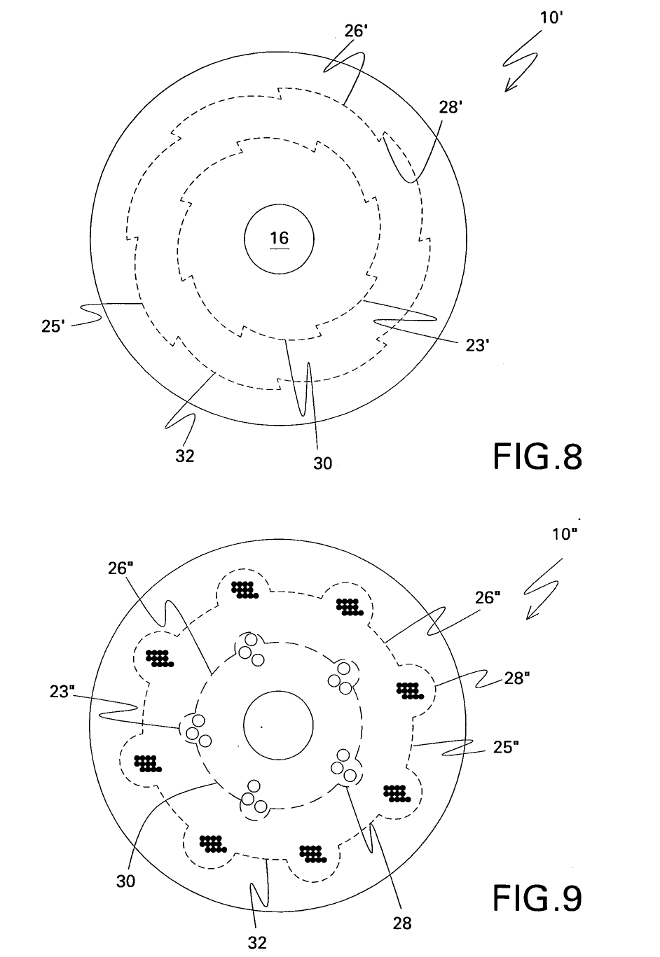

FIG. 8 illustrates a microfluidic sampler 10′ which is similar to microfluidic sampler 10 except in the arrangement of the micropillars 30. As shown, the micropillars 30, 32 are arranged in concentric saw-toothed configurations. Specifically, a first saw-toothed configuration of micropillars 30 makes up a first set of rings 23′, and a second saw-toothed configuration of micropillars 32 makes up a second set of rings 25′. It should be appreciated that each set of rings 23′, 25′ may be formed of one or more separate rings of micropillars 30, 32. The saw-toothed configuration is formed so as to take account of the direction of spin of the microfluidic sampler 10′. In this way, larger agents can skip along the filtering zones 26′ to reside in the detection zones 28′, allowing smaller agents to filter through the first set of rings 23′ toward the second set of rings 25′.

FIG. 9 illustrates another microfluidic sampler 10″ which is similar to the microfluidic samplers 10, 10′ except in the arrangement of the micropillars 30. As shown, the micropillars 30, 32 are arranged in concentric rings that include a plurality of coves. Specifically, a first grouping of micropillars 30 makes up a first set of rings 23″, and a second grouping of micropillars 32 makes up a second set of rings 25″. Each of the sets of rings 23″, 25″ includes a plurality of filtering zones 26″ separated by detection zones 28″. The detection zones 28″ in the first set of rings 23″ may be offset from the detection zones 28″ in the second set of rings 25″. It should be appreciated that each set of rings 23″, 25″ may be formed of one or more separate rings of micropillars 30, 32. In this way, larger agents can skip along the filtering zones 26″ to reside in the detection zones 28″, allowing smaller agents to filter through the first set of rings 23″ toward the second set of rings 25″.

It should be appreciated that the micropillars 30, 32 may take any of various configurations or profiles. For example, the micropillars 30, 32 may be round, oblong or oval, square, diamond-shaped, trapezoidal, polygonal such as rectangular or octagonal, or any other suitable shape. Some shapes may be advantageous for certain tasks. For example, trapezoidal micropillars are adept at assisting larger agents to slide along such shaped micropillars, whereas larger agents may become mired against diamond-shaped micropillars. Thus, trapezoidal micropillars may be advantageously positioned in filtration zones, whereas other shaped micropillars may be used within detection zones.

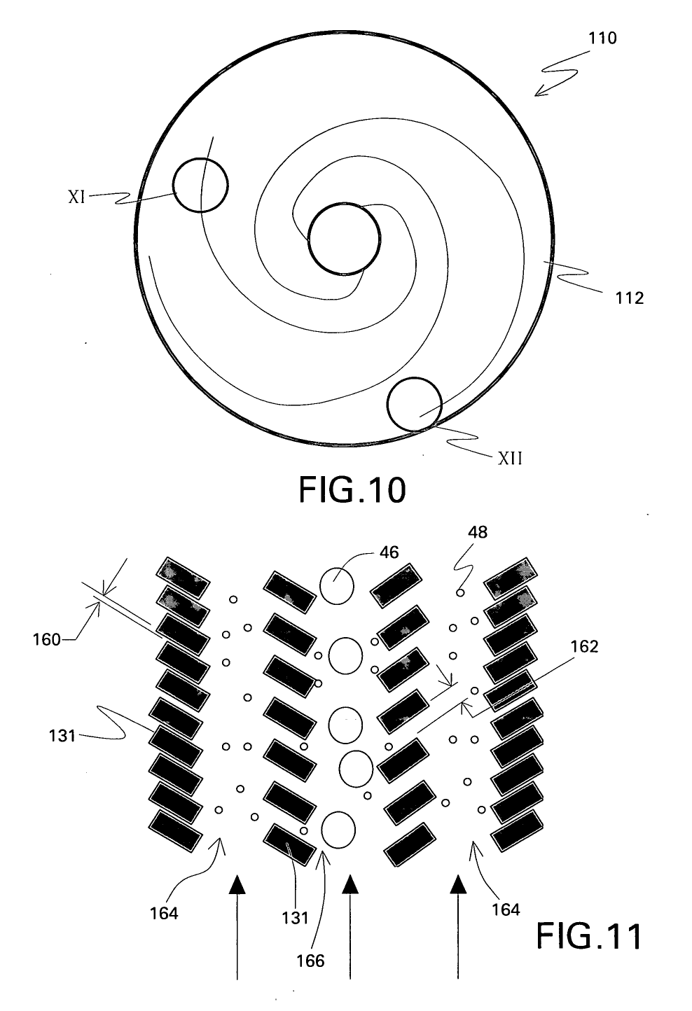

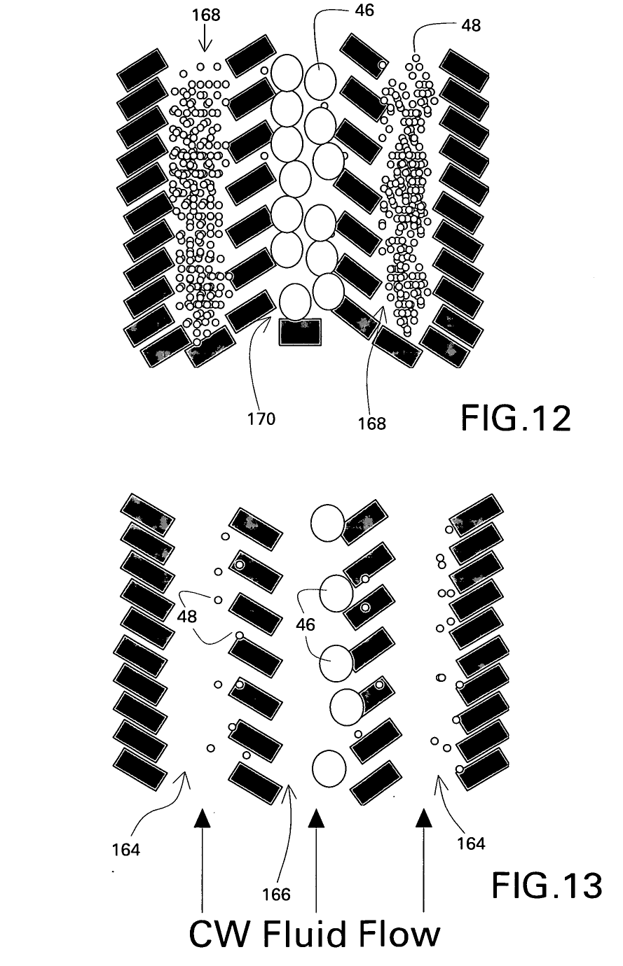

Referring now to FIGS. 10-12, next will be described a microfluidic sampler 110 including a first disc 112. Microfluidic sampler 110 includes micropillars 131 positioned in such a way as to form a plurality of spirally winding and porous microchannels. As shown in FIG. 10, the micropillars 131 are positioned to form alternating porous microchannels 164, 166. The microchannels 164 are each formed with one line of micropillars 131 spaced a first distance 160 apart and a second line of micropillars 131 spaced a second distance 162 apart, while the microchannel 166 is formed with two lines of micropillars 131 spaced a second distance 162 apart. The second distance 162 is sufficiently large or porous to allow particles 48 to migrate into the microchannels 164 while retaining particles 46 within the microchannel 166. Through such an arrangement, coupled with the centrifugal force exerted on the particles 46, 48, a separation of the particles occurs in the microchannels 164, 166. The microchannels 164 lead into first detection zones 168, into which the particles 48 are collected. The microchannels 166 lead into second detection zones 170, into which the particles 46 are collected.

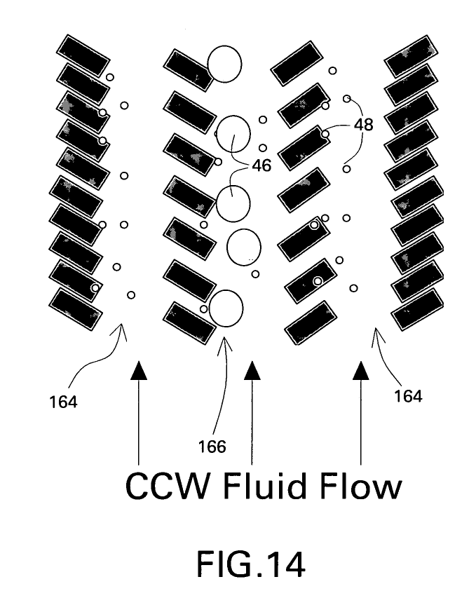

FIGS. 13-14 illustrate the effects of rotational direction on the separation of agents of different species among the porous microchannels 164, 166. Specifically, a clockwise spinning of the microfluidic sampler will allow agents of a smaller size to flow outwardly in greater numbers toward one of the microchannels 164 relative to the other microchannels 164. The same phenomenon can be seen for a counterclockwise spinning of the microfluidic sampler.

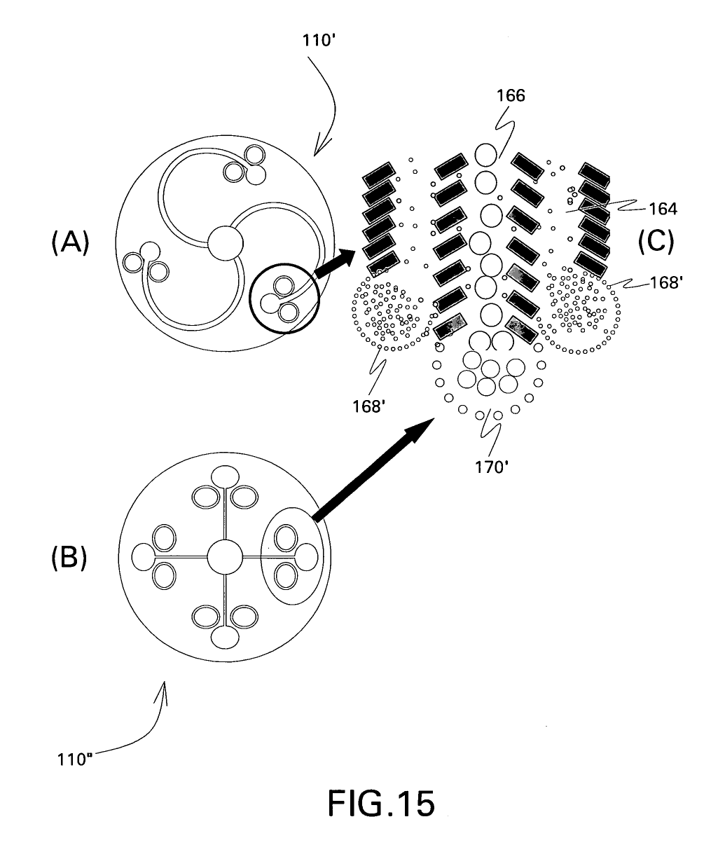

FIGS. 15A-C illustrate various alternative embodiments to the microfluidic sampler 110. Specifically, FIG. 15A shows a microfluidic sampler 110′ that includes spirally winding and porous microchannels as in microfluidic sampler 110. FIG. 15B shows a microfluidic sampler 110″ that includes microchannels extending radially outwardly, each shown to be orthogonal to the others. FIG. 15C illustrates the microchannels 164, 166. The microchannels 164 extend toward a detection zone 168′ which is made up of micropillars forming a pocket into which agents of a specific species can accumulate. The microchannels 166 extend toward a detection zone 170′ which is formed of micropillars making up a pocket into which agents of a different specific species can accumulate.

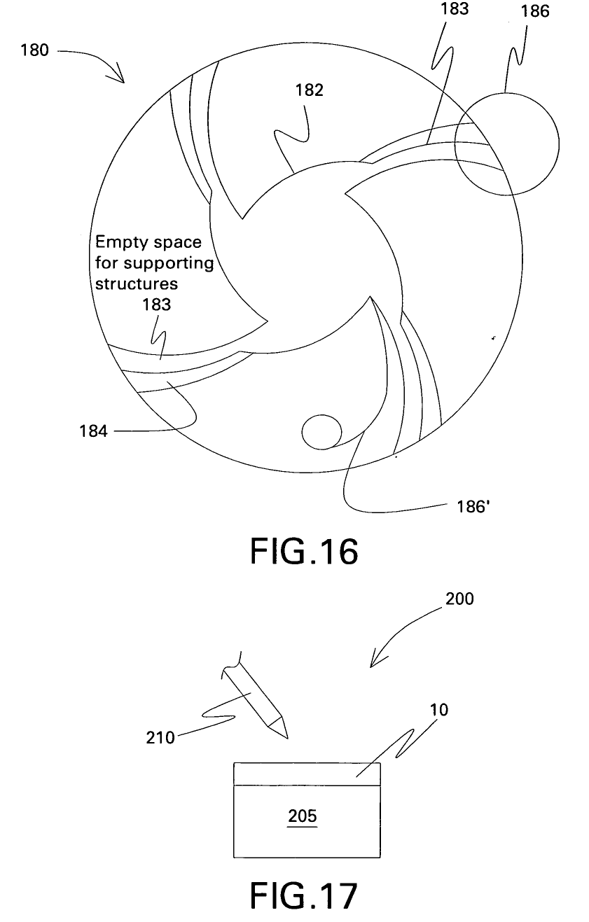

FIG. 16 illustrates a microfluidic sampler 180 that includes spirally winding microchannels such as those illustrated in FIGS. 10, 11, 15A and 15C. Two porous microchannels 183, 184 are shown in FIG. 16. It should be appreciated that more porous microchannels may be formulated on the microfluidic sampler 180 than is shown. A line of micropillars 182 is positioned to prevent any agent species from entering the microchannels 184 from an interior position in the disc. The micropillars 182 may be trapezoidal in shape, to enhance the ability of agents to slide along the micropillars 182 into the microchannels 183. Once in the microchannels 183, the centrifugal force, coupled to the porous nature of the walls separating the microchannels 183 and 184 will allow the smaller agent species to migrate from the microchannels 183 into the microchannels 184, thereby enhancing the separation of larger agent species from smaller agent species into separate detection zones 186. Depending upon rotational speed, the microchannels 183, 184 may spiral to a greater degree toward separate detection zones 186′.

FIG. 17 illustrates an analyzing system 200 that includes a rotating mechanism 205 and a detector 210. A liquid containing agents to be analyzed is collected in a sampling vial that contains antibody-conjugated microbeads. This allows fast homogeneous mixing and a rapid, selective capture of agents onto the microbeads. It should be appreciated that the agents may have been in a dry or powdered form and added to a liquid to allow for its analysis.

This fluidic medium is then introduced to a microsampler disc 10, which is placed on the rotating mechanism 205. The rotating mechanism 205 rotates the microfluidic sampler 10 to impart centrifugal force onto the agents, thereby separating them according to their respective sizes at specific radial positions on the disc surface. Then, in one embodiment, antibody-labeled surface enhanced Raman spectroscopy (SERS) tags are released to wash across the disc surface. The agents captured on the immobilized beads will bind with specific SERS tags for detection by a Raman spectrometer. By measuring the Raman response from specific areas on the disc, multiple biological threat agents can be detected and identified. The detector 210 may be any suitable mechanism for detecting agent species. For example, for the above-described embodiment, a Raman spectrometer may be used as the detector 210. For other embodiments, other suitable detectors may be used. For example, if the agents species are tagged with fluorescents, then a suitable detector 210 may be a fluorometer with a laser or other light source. Other suitable detectors 210 may be colorimeters that measure absorbance, reflectance or just visual change of color and a single electroluminescence. In summary, the type of detector to be used is dependent on the type of assays and more specifically the signals generated from the tagged target.

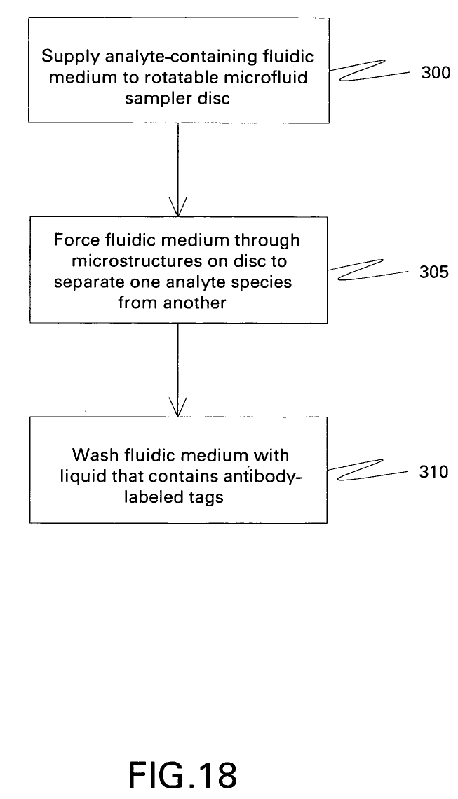

Next, with specific reference to FIG. 18, will be described the process of separating out one agent species from another. At Step 300, a fluidic medium containing two or more species of agents is supplied to a microsampler disc. The microsampler disc, such as disc or plate 12, includes a plurality of microstructures for filtering at least one species of agent from other species of agents. The microstructures may include micropillars within one or more microchannels and micropillars positioned in rings and having different sizes. At Step 305, a force is exerted on the microsampler disc to promote movement of the fluidic medium through the microstructures to induce separation of one species of agent from other species of agents. The force may be centrifugal force induced by rotation of the microsampler disc. At Step 310, the disc is washed with a liquid containing antibody-labeled tags for binding with specific species of agents captured on the immobilized microbeads.

While the-invention has been described in detail in connection with only a limited number of embodiments, it should be readily understood that the invention is not limited to such disclosed embodiments. Rather, the invention can be modified to incorporate any number of variations, alterations, substitutions or equivalent arrangements not heretofore described, but which are commensurate with the spirit and scope of the invention. For example, while the embodiments of the invention have described a single microfluidic sampler to be analyzed, it should be appreciated that suitable analysis systems may include a rotation mechanism capable of rotating a plurality of microfluidic samplers at one time. Such a rotation mechanism may incorporate a plurality of rotating shafts spaced apart, or may have a plurality of rotating sections into which individual microfluidic samplers may be placed. Additionally, while various embodiments of the invention have been described, it is to be understood that aspects of the invention may include some of the described embodiments. Accordingly, the invention is not to be seen as limited by the foregoing description, but is only limited by the scope of the appended claims.

What is claimed as new and desired to be protected by Letters Patent of the United States is:

1. A microsampler plate for use in the detection of agents, comprising a plurality of microstructures configured and spaced to promote movement of a fluidic medium containing agents radially outwardly and to promote filtering of one species of agents from other species of agents.

2. The microsampler plate of claim 1, wherein said microstructures comprise at least one concentric set of micropillars configured to form a plurality of filter zones sequentially interspersed among a plurality of detection zones.

3. The microsampler plate of claim 2, wherein each said detection zone comprises a first substantially straight line of micropillars and a second substantially straight line of micropillars, said first and second substantially straight lines of micropillars abutting one another and angling away from each other at an abutment point.

4. The microsampler plate of claim 2, wherein said at least one concentric set of micropillars comprises two or more concentric sets of micropillars.

5. The microsampler plate of claim 2, wherein each of said at least one concentric set of micropillars comprises two or more concentric rings of micropillars.

6. The microsampler plate of claim 5, wherein said two or more concentric rings of micropillars comprise:
a first concentric ring of first micropillars, each having at least one of a first size and a first spacing between adjacent said first micropillars, wherein either or both of said first size and said first spacing are chosen to capture a first species of agent and filter out a second species of agent; and
a second concentric ring of second micropillars, each having at least one of a second size and a second spacing between adjacent said second micropillars, wherein either or both of said second size and said second spacing are chosen to capture said second species of agent.

7. The microsampler plate of claim 2, wherein said plurality of microstructures further comprise a reservoir and at least one microfluidic channel in fluid communication with said reservoir and leading in a direction of said at least one concentric set of micropillars.

8. The microsampler plate of claim 7, wherein said at least one microfluidic channel comprises micropillars interspersed in a pattern to facilitate pre-filtering of two or more species of agents.

9. The microsampler plate of claim 8, wherein said pattern facilitates pre-filtering by enhancing the movement of a first species of agent along a first pathway within said microfluidic channels and the movement of a second species of agent along a second pathway within said microfluidic channels, said first and second pathways angling away from each other in a direction from said reservoir to said at least one concentric set of micropillars.

10. The microsampler plate of claim 1, wherein said microstructures comprise at least one set of micropillars configured in a saw-toothed arrangement to form a plurality of filter zones sequentially interspersed among a plurality of detection zones.

11. The microsampler plate of claim 1, wherein said microstructures comprise at least one concentric set of micropillars having a plurality of coves, each forming a detection zone, sequentially interspersed among a plurality of filter zones.

12. The microsampler plate of claim 1, wherein said plurality of microstructures comprises a plurality of micropillars arranged to form at least two porous microchannels.

13. The microsampler plate of claim 12, wherein said plurality of micropillars comprise:
a first set of micropillars positioned into n number of lines of micropillars to form n−1number of microchannels tracing a substantially spiral path radially outwardly; and
a second set of micropillars positioned to form n−1number of detection zones.

14. The microsampler plate of claim 13, wherein said micropillars in said first set of micropillars are spaced and positioned so as to promote filtering of one species of agents into one of said n−1number of microchannels.

15. The microsampler plate of claim 14, wherein said micropillars in said second set of micropillars are spaced and positioned so as to promote collection of one species of agents into one of said n−1number of detection zones.

16. A microsampler device, comprising:
a microsampler plate comprising:
a concentric set of first micropillars, wherein each said first micropillar has at least one of a first size and a first spacing between adjacent said first micropillars; and
a concentric set of second micropillars, wherein each said second micropillar has at least one of a second size and a second spacing between adjacent said second micropillars; and
a second plate positioned over said microsampler plate and resting upon said concentric sets of first and second micropillars.

17. The microsampler device of claim 16, wherein said second plate includes an annular wall defining a reservoir into which fluid containing one or more species of agents may be introduced to the microsampler device.

18. The microsampler device of claim 17, wherein said microsampler plate comprises an opening configured to receive a rotatable shaft for translating a rotational force to the microsampler device.

19. The microsampler device of claim 18, wherein said microsampler plate comprises at least one microfluidic channel in fluid connection with said reservoir and leading in a direction of said concentric set of first micropillars.

20. A system for analyzing analytes in a fluidic medium, comprising:
a microsampler device comprising a microsampler plate having a plurality of microstructures for filtering a first species of agents from other species of agents;
a rotational mechanism for rotating the microsampler device to exert a centrifugal force on the fluidic medium; and
a detector for detecting the presence of one or more specific species of agents.

21. The system of claim 20, wherein said microstructures are configured and spaced to promote movement of a fluidic medium containing agents radially outwardly.

22. The system of claim 20, wherein said microstructures comprise at least one concentric set of micropillars configured to form a plurality of filter zones sequentially interspersed among a plurality of detection zones.

23. The system of claim 22, wherein said microstructures further comprise a reservoir and at least one microfluidic channel extending outwardly from said reservoir in a direction toward said at least one concentric set of micropillars.

24. The system of claim 23, wherein said at least one microfluidic channel comprises micropillars interspersed in a pattern to facilitate pre-filtering of two or more species of agents.

25. The system of claim 19, wherein said microstructures comprise:
a first set of micropillars positioned into a number of lines of micropillars to form a plurality of porous microchannels; and
a second set of micropillars positioned to form a plurality of detection zones.

26. The system of claim 25, wherein said micropillars in said first set of micropillars are spaced and positioned so as to promote filtering of one species of agents into one of said plurality of porous microchannels.

27. The system of claim 26, wherein said micropillars in said second set of micropillars are spaced and positioned so as to promote collection of one species of agents into one of said plurality of detection zones.

28. The system of claim 20, wherein said microsampler device comprises a second plate positioned over said microsampler plate and resting upon at least a portion of said microstructures.

29. A method of detecting at least one species of agent within a fluidic medium, comprising:
introducing a fluidic medium containing at least one species of agents to a microsampler plate, the microsampler plate comprising a plurality of microstructures for filtering said at least one species of agents from the remainder of the fluidic medium;
rotating the microsampler plate to promote movement of the fluidic medium outwardly;
collecting the at least one species of agents in a specific set of detection zones; and
analyzing the at least one species of agents.

30. The method of claim 29, wherein said introducing comprises introducing the fluidic medium into a reservoir that feeds into at least one microfluidic channel extending toward at least one set of micropillars.

31. The method of claim 30, further comprising pre-filtering one of said at least one species of agents from other of said at least one species of agents.

32. The method of claim 31, wherein said pre-filtering comprises rotating the microsampler plate to promote movement of the fluidic medium through said at least one microfluidic channel, said at least one microfluidic channel comprising a plurality of micropillars in a pattern that enhances the movement of a first species of agents along a first pathway within said at least one microfluidic channel and the movement of a second species of agents along a second pathway within said at least one microfluidic channel.

33. The method of claim 30, wherein said collecting comprises filtering out other species of agents from said at least one species of agents.

34. The method of claim 33, wherein said filtering out comprises deflecting other species of agents from said detection zones.

35. The method of claim 29, wherein said rotating comprises rotating said microsampler plate in at least one of a clockwise direction and a counter-clockwise direction.

36. The method of claim 29, wherein said analyzing comprises adding a tag to said at least one species of agents.

37. The method of claim 36, wherein said analyzing comprises directing a light source at said at least one species of agents.

38. The method of claim 29, wherein said introducing comprises introducing the fluidic medium into a reservoir that feeds into a plurality of microfluidic channels extending substantially spirally outwardly.

39. The method of claim 38, wherein said plurality of microfluidic channels extend to a plurality of detection zones.
